(19) United States
(12) Patent Application Publication
(10) Pub. No.: US 2008/0208046 A1
(43) Pub. Date: Aug. 28, 2008

(54) Ultrasonic Diagnostic Imaging System With Integrated Network Analyzer

(75) Inventor: Michael Pierce, Carnation, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/914,269

(22) PCT Filed: May 10, 2006
(86) PCT No.: PCT/IB06/51475

§ 371 Date: Nov. 13, 2007

Related U.S. Application Data
(60) Provisional application No. 60/683,435, filed on May 19, 2005.

(51) Int. Cl. A61B 8/00; H04L 12/26

(52) U.S. Cl. 600/437; 370/245

(57) ABSTRACT

An ultrasound diagnostic imaging system (10) is provided with its own onboard network analyzer (34). When a problem with a network to which the ultrasound system is connected is discovered, the network analyzer is started on the ultrasound system. A test is run by transmitting DICOM data from the ultrasound system to another device on the network. The network analyzer is operable in a listening mode to produce a capture file of the raw packet data concerning the DICOM transmission and to present the capture file to a user for analysis such as by parsing out the DICOM communication data on the network.

This invention relates to medical diagnostic ultrasound systems and, in particular, to diagnostic ultrasound systems with onboard network monitoring and analysis capabilities.

Ultrasound systems are generally designed and built as highly portable instruments. Ultrasound systems may be configured as cart-borne instruments or as portable instruments the size of laptop or tablet computers which can be carried to a patient's bedside. While this portability means that ultrasound systems can be moved around a hospital or clinic, there is often a desire to connect an ultrasound system to a network so that its digital images can be transmitted to another location electronically, usually for storage or review. U.S. Pat. No. 5,715,823 (Wood et al.) illustrates an ultrasound system which can be connected to the Internet, whereby images acquired by the system can be accessed and sent worldwide. Since large image files such as live image loops utilize a large amount of digital storage it is frequently desirable in a large institutional setting such as a hospital or clinic to store these images on a separate device such as a picture archiving and communication system (PACS). In order to make this possible the ultrasound system needs to have connectivity capability so that it can interface successfully and easily to the network of the hospital or clinic.

At times, however, it can develop that the ultrasound system does not successfully communicate over the network of hospital or clinic. This may be due to hardware incompatibility, but more likely is due to and incompatibility of the protocols and exchanges on the network necessary to establish a valid connection with the network setups of the ultrasound system. These difficulties can arise when the ultrasound system is first connected to the network and the incompatibility manifests itself, or at a later time when changes are made to the network or other devices on the network. In such instances a serviceperson for the ultrasound system may be called in to remedy the difficulty. The first action that the serviceperson may request is to connect a network analysis device to the network to try to analyze the problem. But hospitals and clinics are generally reluctant to allow such connections. This is because these networks are usually complex and contain sensitive medical and personal patient data which the facility does not want to compromise. There is also a concern over viruses and other possibly deleterious effects from the access of unknown software to a network. Thus, bringing analysis instrumentation and devices to the hospital network can pose difficulties for both the network administrators trying to maintain the security of their network and data, as well as the serviceperson trying to resolve the ultrasound system network connection.

In accordance with the principles of the present invention, an ultrasonic diagnostic imaging system is provided which contains its own onboard network analysis capability. When a network connection problem arises, this capability can be used to monitor network traffic at the network level from the ultrasound system without the need to attach any other instruments to the network. Data traffic on the network can be monitored and selectively captured, then analyzed to locate the source of a network problem involving the ultrasound system. The system is particularly useful when the ultrasound system needs to transmit or receive DICOM format data over the network.

In the drawings:

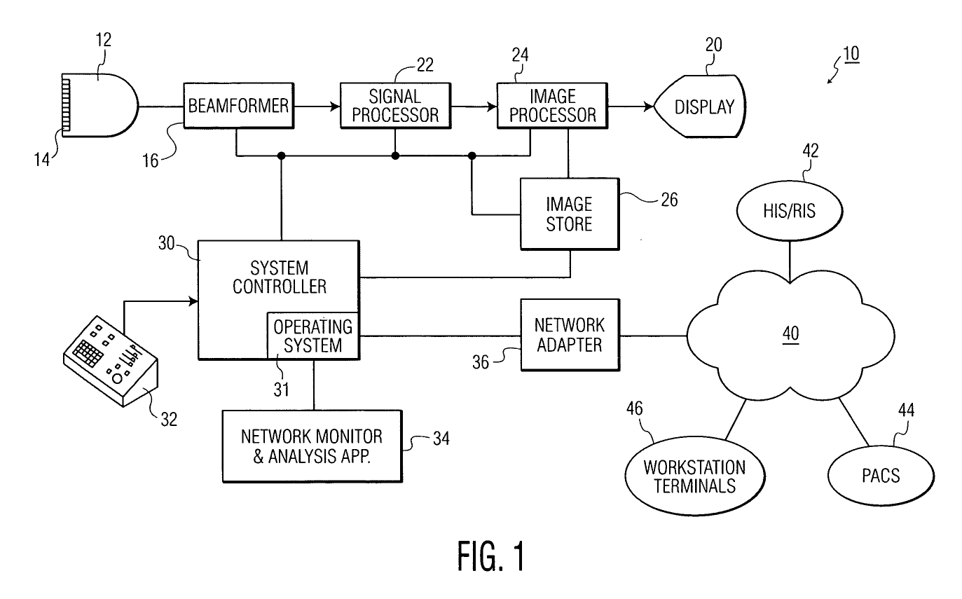

FIG. 1 illustrates in block diagram form an ultrasonic diagnostic imaging system and network constructed in accordance with the principles of the present invention.

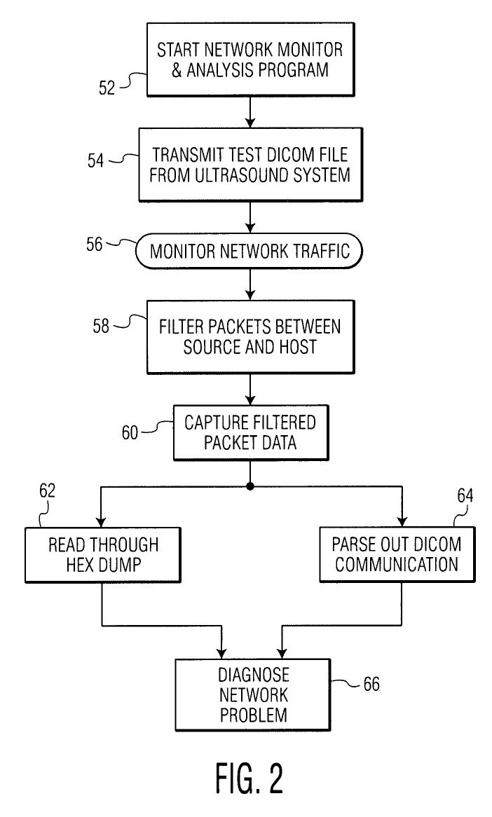

FIG. 2 is a flowchart of the analysis of a DICOM network communication problem in accordance with the present invention.

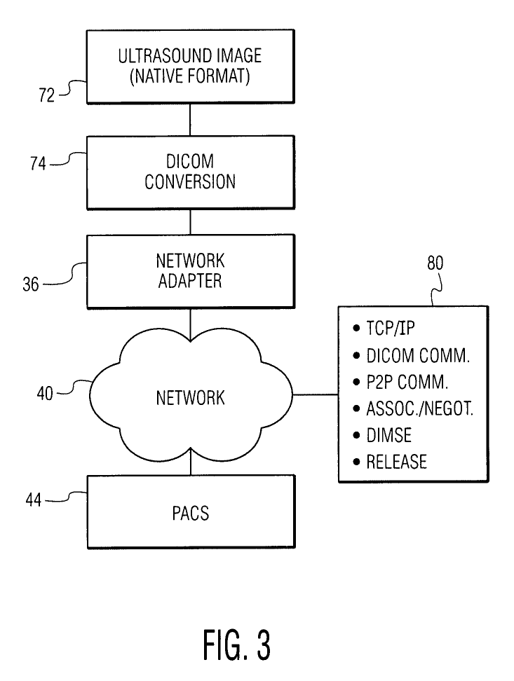

FIG. 3 illustrates the sequence of events in a typical network transfer of a DICOM file.

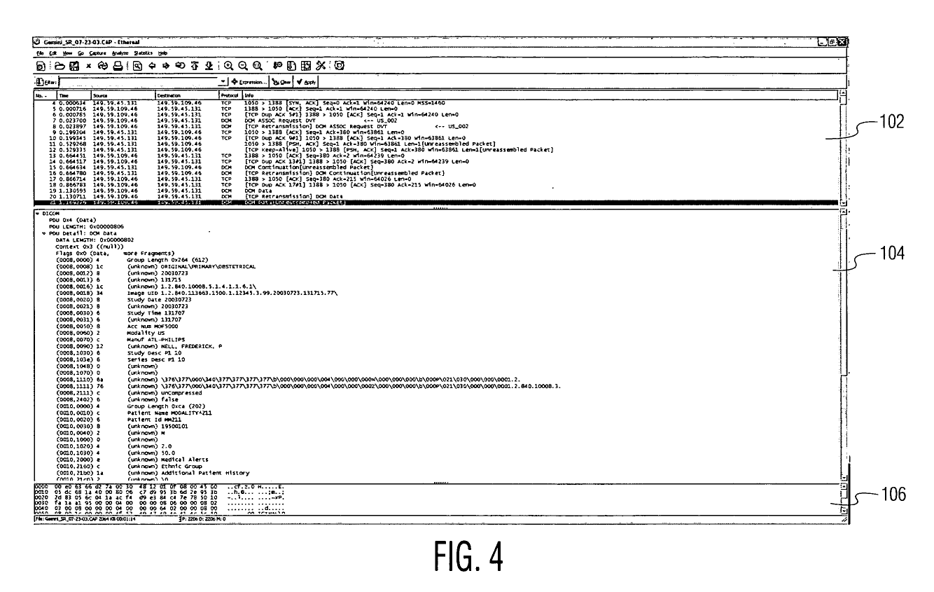

FIG. 4 illustrates the capture of DICOM packet traffic on a network.

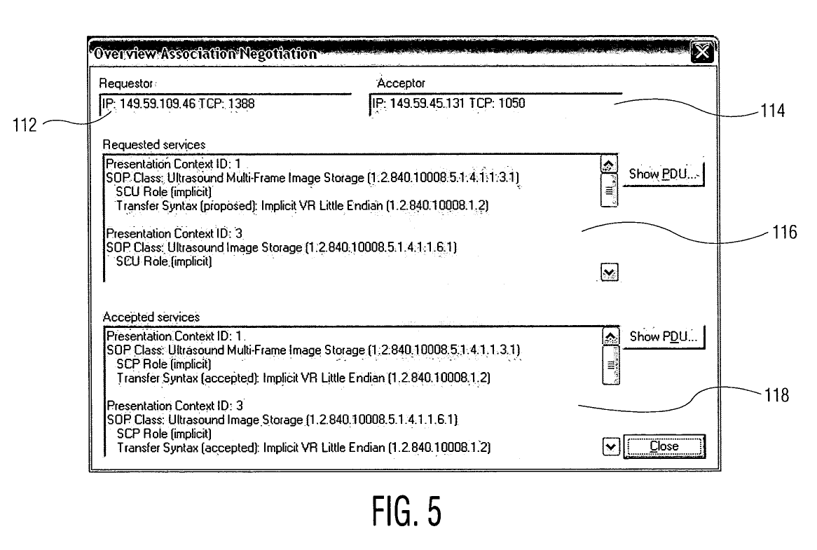

FIG. 5 illustrates captured association/negotiation network communication.

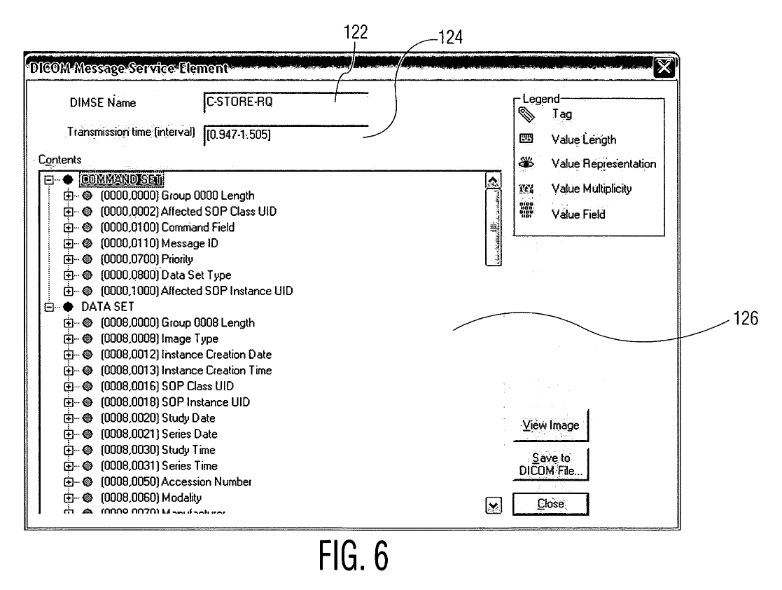

FIG. 6 illustrates captured DICOM network service element data.

Referring first to FIG. 1, an ultrasonic diagnostic imaging system 10 and network constructed in accordance with the principles of the present invention is shown in block diagram form. The ultrasound image acquisition, processing and display path of the ultrasound system 10 starts with an array probe 12 having an array of transducer elements 14. The transducer array transmits ultrasonic waves under control of a beamformer 16 and receives echo signals from the subject being imaged which are converted to electrical signals. The signals received by the individual elements of the array are appropriately delayed and combined by the beamformer 16 to form coherent echo signals. The echo signals may then undergo specific processing for the type of information acquired and to be displayed such as detection, filtering, Doppler processing, harmonic signal separation, and the like. This processing is performed by a signal processor 22. The processed signals are formed into an image of a desired display format by an image processor 24 and the processed images are displayed on an image display 20. The images may be stored in an image store 26 for further processing and review or later display.

The functioning of the processes of the image acquisition, processing and display path is controlled and coordinated by a system controller 30 which is coupled to the elements of the signal path. The system controller responds to commands from a user which can be input by a graphical user interface on a display or from a control panel 32 or voice recognition system. The system controller runs an operating system (OS) 31 which performs functions involving the user interface and/or the display 20. In accordance with the principles of the present invention the OS can also run a network monitor and analysis application 34 which is generally stored on a disk drive or other storage medium. The network monitor and analysis application can be one of a variety of available applications, the choice of which depends upon the OS being used and other operational considerations. Suitable applications include tcpdump for UNIX platforms, WinDump and WinPcap for Windows platforms, Ethereal, libpcap, and others, many of which are freely available downloads. The OS 31 is coupled to a network adapter 36 by which the ultrasound system communicates over a network 40. The network adapter comprises hardware and software by which the ultrasound system 10 can communicate over the network 40, formats for which include Ethernet, FDDI, PPP, token-ring, IEEE 802.11, I²C and others. Generally the network adapter will be in the form of a network interface card (NIC) or a modem card. When the ultrasound system is connected to the network 40 it can communicate with other devices on the network, examples of which include hospital information/radiology information systems (HIS/RIS) 42, picture archival and communication systems (PACS) 44, and workstation terminals 46.

When the ultrasound system 10 is first connected to the network 40 or while connected to the network 40, difficulty may arise with some or all of the communications between the ultrasound system and another device or devices on the network. For example, the ultrasound system image processor 24 may format images and other information in the DICOM format. DICOM is a well accepted format for diagnostic images and other medical information and ultrasound images are frequently encoded and stored in the DICOM format. In the arrangement of FIG. 1 it may be desirable to store DICOM formatted images on the PACS system 44, for example. If the network communication for the storage of DICOM information is unsuccessful the network monitor and analysis application may be used to resolve the network communication problem.

An ultrasound system which is constructed to send and receive data over a TCP/IP network such as that shown in the above referenced Wood et al. patent operates on blocks of data called packets. Each packet of data on the network has headers which identify certain characteristics of the packet such as the device which was the source of the packet, the device which is the destination of the packet, the type of data, and others. Network monitor and analysis programs monitor the flow of packets on the network and record or capture them in their raw form. For instance, suppose that the Ethernet card of the ultrasound system picks up a packet from the network. The packet is passed to the OS and the OS must determine what type of packet has been received. The OS does this by stripping off the Ethernet header of the packet and looking at the next layer. Suppose the packet is found to be an IP packet. The OS then strips off the IP header to determine what type of IP packet it is. Suppose that the OS finds that this is a UDP (User Datagram Protocol) packet. The OS then strips off the UDP header and hands the packet over to the application for which the packet is intended. If the packet is now analyzed, little can be learned about its communication over the network because the headers have been removed. The purpose of a network monitor and analyzer of the present invention is to capture these packets with their headers intact so that their communication over the network and effects of other network devices can be studied. To do this the capture system needs to bypass the protocol stack of the ultrasound system and access the raw data traffic on the network, interacting directly with the network interface.

A network monitor and analysis package could capture all of the packet traffic that transits the network but preferably it exercises some selectivity about the data it acquires, a process known as packet filtering. A packet filter compares an incoming packet with criteria predefined by an operator and determines whether the packet should be accepted and copied to the listening application. In this way the listening application and its operator are not overwhelmed by a flood of data, but only see a subset of the network traffic that may be of interest. For example a filter could be set to capture only the ftp traffic generated by a particular host such as the PACS system 44. As other examples, the packet filter could be set to pick up all UDP packets, or all IP packets with a certain value in the protocol type field. By using a properly adjusted packet filter the operator can quickly zero in on a particular problem device or type of communication.

A second characteristic of the monitor and analysis package which is significant is the buffering of the captured packets. When a packet is acquired it is stored in a buffer together with other useful information such as a receipt timestamp and the size of the packet. A buffer allows packets on a high data rate network to be quickly stored and a larger buffer allows a significant number of packets to be acquired before being transferred to an application such as an analysis program. Buffers employed for this purpose are generally circular, meaning that data must be transferred out of the buffer before it is full, whereupon data will be overwritten and lost.

Two other characteristics of a monitor and analysis program may be useful in certain instances. One is packet injection, by which a user is able to write raw packets to the network. When this feature is present the user is able to send a customized packet with user-defined headers over the network, a feature useful to diagnose a specific network problem. Often this feature permits the same packet to be sent repeatedly at a high data rate to generate high speed traffic for testing purposes. In a purely listening run as described below, packet injection is not used.

The other characteristic which a monitor and analysis package may have is a network monitoring capability. This capability enables the program to calculate simple statistics on network traffic. A network monitor can classify network traffic using the same principles as the packet filter, then counts the number of packets of the measured classification. This information is passed to the user at selected regular intervals. A network monitor could be used to determine the number of DICOM packets that transit the network in a given interval, or the percentage of network traffic that is DICOM packets, for instance.

Thus, a typical monitor and analysis program of the present invention will be able to capture raw packets transiting the network, both those destined to and from the ultrasound system and others exchanged by other hosts on the network. It should be able to filter the packets according to user-specified rules before passing them on to the analysis application. It may optionally be able to transmit raw packets to the network and it may optionally be able to gather statistics about the network traffic.

After the captured network data has been transferred to the analysis application it must be displayed in a way that is useful to the network diagnostician. This is done by giving the application the ability to dissect and display information of a wide variety of communication protocols. The Ethereal analysis program can dissect 673 commonly used protocols, for example. A number of analysis applications are capable of reading capture file formats of many of the other more widely used “sniffer” programs in addition to their own. This provides the ability to read live network data as well as data previously acquired by other capture programs.

An example of a method of using a monitor an analysis program in a listening mode for the analysis of a DICOM network communication problem is illustrated by the flowchart of FIG. 2. The user begins at 52 by starting the network monitor and analysis program 34 on the ultrasound system. When the program is running and in a condition to monitor network traffic, the user transmits a test DICOM file at 54 from the ultrasound system to another device on the network, such as one with which there is a communication problem. The network monitor and analysis program then monitors the network traffic at 56.

FIG. 3 illustrates the transmitting and monitoring steps of FIG. 2 in greater detail for a DICOM test. An ultrasound image is acquired by an ultrasound system in an image format which is native to that system as indicated at 72. The image may be stored in the image store 26 in the native format or may be converted to the DICOM format at step 74 before being stored. When native format storage is used the image is converted to the DICOM format before being stored over the network. The DICOM image is formatted into packets for the communication protocol used by the network and coupled to the network adapter at 36. DICOM image packets are then transmitted to a host device over the network 40. As the box 80 indicates, packets are transmitted in a TCP/IP protocol for DICOM communication and the communication employed is point-to-point (P2P) from the IP address and sending port of the ultrasound system to the IP address and receiving port of a host device such as a PACS system 44. The initial packets in the transmission establish the basic handshake communication between the devices in what is known as association and negotiation, by which the parameters of the communication are established, such as whether the receiving host can process DICOM data and so forth. Once the association/negotiation has been completed the DICOM message service elements (DIMSE) are exchanged, where DIMSE refers to DICOM elements in general. After the exchange of DIMSE has been completed a release is effected, ending the particular communication of DICOM data.

Returning to FIG. 3, packets exchanged between the ultrasound system and another host device on the network are filtered at 58, as by filtering packets with the IP addresses of the source and the host. The filtered packet data is captured in a capture file at 60. The capture file can be displayed to the user so that the user can read through a hex dump of the data as indicated at 60. Another alternative is to parse out only selected information with the analysis program such as parsing out only the DICOM communication. The user can then diagnose the network problem at 66.

FIG. 4 illustrates a display screen of a viewer showing a typical capture file of network DICOM data from a constructed embodiment of the present invention. In the upper window 102 of the viewer each captured packet is listed on a separate line of the window. The packet information includes the relative time at which the packet was captured, the IP address of the source, an ultrasound system in this example, and the IP address of the host device (Destination) with which the ultrasound system was communicating. The protocol column indicates the type of packet, TCP for the first packet. The first packet contains the notations “[SYN, ACK]”, which indicate that this packet was involved in the handshaking (association/negotiation) between the ultrasound system and the host device. The protocol entry DCM is the identifier for a DICOM packet which has been parsed out by this viewer. In the packet sequence shown in window 102 the host device is responding to a successful storage request, informing the source device (the ultrasound system) the parts of the request to which it can and cannot comply.

The bottom packet #21 in the upper window 102 is highlighted, causing the details of packet #21 to be displayed in the middle window 104. The identifier shows that packet #21 is a DICOM (DCM) packet and the packet detail shows the detail expected of a DICOM packet such as patient demographic information, imaging system modality, physician, date of study, and so forth.

The lower window 106 shows the packet data expressed in hexadecimal form. Each byte of the packet is expressed as two consecutive hexadecimal digits. The hexadecimal data illuminates the packet data in its most basic machine language form.

FIG. 5 illustrates another typical viewer of a constructed embodiment of the present invention which displays association/negotiation information from a capture file. The upper two windows 112 and 114 display the IP addresses and protocol information of the two hosts involved in the handshake, the requester and the acceptor. The windows 116 and 118 split out the services requested by the requester and the services accepted by the acceptor in the course of the handshake. This viewer is seen to provide a view of parsed out upper layer of a DICOM application communication, providing information of an association/negotiation request at a higher level than the previously illustrated viewer. A release request at the conclusion of a communication will have similar data.

FIG. 6 illustrates a viewer of another embodiment of the present invention which shows a parse of the DIMSE data, the substance of the DICOM communication. The window 122 of the viewer provides the DIMSE name and the window 124 provides the time interval during which the packet was transmitted. The window 126 illustrates the individual components of the DIMSE with their DICOM data tags and representations.

What is claimed is:

1. An ultrasound diagnostic imaging system which may be connected to a network for the exchange of ultrasound information with another device on the network comprising:
an operating system resident on the ultrasound system;
a network adapter by which the ultrasound system is connected to the network; and
a network analysis application resident on the ultrasound system and operable by the operating system to capture raw network packet data or display a network capture file.

2. The ultrasound diagnostic imaging system of claim 1, wherein the network adapter comprises one of an Ethernet, FDDI, PPP, token-ring, or IEEE 802.11 network interface.

3. The ultrasound diagnostic imaging system of claim 1, wherein the network analysis application is operable to both capture raw network packet data and to display the captured packet data to a user in a capture file.

4. The ultrasound diagnostic imaging system of claim 1, wherein the network analysis application is operable to capture DICOM packet data employing a TCP/IP protocol.

5. The ultrasound diagnostic imaging system of claim 4, wherein the network analysis application displays a capture file of a DICOM message service element.

6. The ultrasound diagnostic imaging system of claim 5, wherein the DICOM message service element comprises a DICOM image file.

7. The ultrasound diagnostic imaging system of claim 5, wherein the network analysis application further comprises means for parsing out a DICOM communication.

8. The ultrasound diagnostic imaging system of claim 1, wherein the network analysis application includes a packet filter.

9. The ultrasound diagnostic imaging system of claim 8, wherein the network analysis application displays a capture file with a packet timestamp and a host IP address.

10. The ultrasound diagnostic imaging system of claim 8, wherein the network analysis application further includes a capture file buffer.

11. A method for diagnosing a network connection to an ultrasound diagnostic imaging system from the ultrasound system comprising:
starting a network monitor and analysis program from the ultrasound system;
transmitting a data file from the ultrasound system to a host device on a network;
monitoring network traffic associated with the data file; and
producing a capture file from at least a subset of the monitored network traffic.

12. The method of claim 11, wherein transmitting further comprises transmitting a DICOM file from the ultrasound system to a host device on a network; and
wherein monitoring further comprises monitoring network traffic associated with the DICOM file.

13. The method of claim 11, wherein monitoring further comprises monitoring raw packet data transiting the network.

14. The method of claim 11, further comprising filtering packet data traffic in accordance with one or more user-defined characteristics.

15. The method of claim 12, further comprising parsing out DICOM communication.

16. A method of diagnosing a network problem comprising:
connecting a network adapter of an ultrasound system to a network;
determining that a communication problem exists between the ultrasound system and at least one device on the network;
acquiring a capture file of network traffic with the ultrasound system while the ultrasound system is connected to the network; and
analyzing the capture file to resolve the communication problem.

17. The method of claim 16, further comprising filtering the network traffic with a packet filter.

18. The method of claim 16, further comprising transmitting packets from the ultrasound system to another device on the network.

19. The method of claim 18, wherein transmitting packets further comprises transmitting packets of a DIMSE element,
wherein the capture file is acquired during and after the transmitting of DIMSE element packets.
